(19) United States
(12) Patent Application Publication
(10) Pub. No.: US 2024/0118198 A1
(43) Pub. Date: Apr. 11, 2024

(54) NEAR INFRARED-II REGION FLUORESCENT RARE EARTH NANOPROBE TEST STRIP AND ITS PREPARATION METHOD

(71) Applicant: Shandong Laboratory of Yantai Drug Discovery, Yantai (CN)

(72) Inventors: Zhen Cheng, Yantai (CN); Zhaorui Song, Yantai (CN)

(21) Appl. No.: 18/456,491

(22) Filed: Aug. 27, 2023

(30) Foreign Application Priority Data
Oct. 9, 2022 (CN) 202211229850X

(51) Int. Cl. G01N 21/3577; C09K 11/02; C09K 11/77; G01N 21/64

(52) U.S. Cl. CPC G01N 21/3577; C09K 11/025; C09K 11/7773; G01N 21/6428

(57) ABSTRACT

A near infrared-II region (NIR-II) fluorescent rare earth nanoprobe (RENP) test strip and its preparation method are disclosed. The NIR-II fluorescent RENP test strip includes a sample pad, a conjugation pad, a nitrocellulose (NC) membrane, an absorbent pad and a plastic backing. The sample pad, conjugation pad, NC membrane, absorbent pad are superimposed on the plastic backing successively along a horizontal direction. Detection antibodies labeled RENPs are immobilized on the conjugation pad; capture antibodies set as a test line and quality control antibodies set as a control line are sprayed on the NC membrane. RENPs with NIR-II luminescence are selected as an efficient fluorescent probe, and its excellent optical properties make the prepared test strip possesses excellent detection sensitivity, good accuracy, high stability and favorable repeatability. Meanwhile, preparation process of test strip is also simple and controllable, which is suitable for scale production.

### TECHNICAL FIELD

The invention relates to a near infrared-II region (NIR-II) fluorescent rare earth nanoprobe test strip and its preparation method, which belongs to the technical field of clinical laboratory medicine.

### BACKGROUND

Sensitive, accurate and convenient determination is essential for the early-stage cancer screening, especially for people living in the suburb and remote areas. The carcinoembryonic antigen (CEA) is taken as an example, as one of the most common tumor biomarkers in clinic, it is a soluble glycoprotein with a relative molecular weight of 150 kilodaltons (KD)-300 KD, and its content in normal human serum is very low. CEA is widely used for screening, diagnosis and prognosis evaluation of various gastrointestinal tumors, such as colorectal cancer, esophageal cancer, gastric carcinoma, and pancreatic carcinoma. CEA can be detected in a patient's serum when the tumor is too small to be examined by imaging technology. The increase of CEA level in serum above the normal value (5 nanograms/milliliter (ng/mL)) generally indicates the occurrence of tumors. Therefore, CEA is mainly applied to the colorectal cancer diagnosis, judging tumor stage, monitoring the progress of tumor disease and treatment, which has displayed important clinical value.

At present, the detection method of tumor biomarkers mainly includes radioimmunoassay (RIA), enzyme linked immunosorbent assay (ELISA), chemiluminescent immunoassay (CLIA), electro-chemiluminescence immunoassay (ECLIA) and lateral flow immunoassay (LFA). RIA has been replaced by other methods because of the radio labelling, cumbersome operations and long time. Other immunoassay methods (ELISA, CLIA and ECLIA) have advantages of high sensitivity and strong specificity, but they are generally used for the automatic detection of large quantities of samples in large hospitals. Gold immunochromatography assay (GICA) has advantages of simple, fast and low cost, but it only can achieve qualitative and semi-quantitative detection.

In recent years, fluorescence lateral flow immunoassay (FLFA) based on nanomaterials (fluorescent dyes, quantum dots and time-resolved fluorescent microspheres, etc.) has been developed rapidly, which has effectively improved the detection sensitivity of LFA. However, these fluorescent materials have some unavoidable defects in luminescence, such as poor optical stability, easy photobleaching and photodegradation. More importantly, the emission wavelength of these fluorescence reporter probes is mostly located in the visible light region (400-700 nanometers (nm)) or near infrared-I region (NIR-I, 700-900 nm). To a certain extent, it can be disturbed by the auto-fluorescence from biological samples. Furthermore, because of the excitation wavelength is close to the emission wavelength, spectra overlap is unavoidable, which further aggravates fluorescence interference. Finally, these probes suffer from weak fluorescence penetration and relatively low quantum yield. The fluorescent labels located inside porous materials are difficult to be detected. All of these factors have severely influence on the signal-to-noise ratio (SNR) of LFA. Therefore, to improve the analytical performances of the conventional LFA platform, it is crucial to develop new fluorescent probes with excellent luminescence properties.

The development of highly sensitive, accurate, convenient and rapid tumor biomarker detection methods has always been a worthwhile goal. Recently, the NIR-II with broadband emission spectra ranging from 1000 nm to 1700 nm has attracted tremendous attentions. Compared to the common fluorescence imaging (400-900 nm), NIR-II fluorescence imaging offers various merits, such as very low tissue autofluorescence and scattering, deeper tissue penetration, and much higher SNR. In numerous NIR-II fluorescent probes, growing interests have been focused on rare earth nanoprobes (RENPs) because of their large Stokes shifts, narrow and multi-peak emission profiles, negligible excitation-emission band overlap, long lifetime and excellent photostability. Particularly, the lack of band overlap and elimination of tissue autofluorescence produces significantly improved S/N ratios. As a highly dynamic research area in molecular imaging field, NIR-II fluorescent probes have been extensively explored in biological imaging, such as living surgical navigation, high resolution fluorescence imaging of deep tissue, and optical imaging of tumor.

However, until now, LFA platform based on NIR-II fluorescent probes have rarely been reported. Considering its outstanding optical properties, it is well worthy of exploring the applications of NIR-II fluorescent probes for LFA in point of care testing (POCT).

### SUMMARY

Aiming at the above problems of the current LFA platform in the prior art, the invention provides a near infrared-II region fluorescent rare earth nanoprobe test strip and its preparation method. By utilizing the luminous advantages of RENPs in the NIR-II, the prepared test strip has the characteristics of accurate quantification, good repeatability, which can meet the needs of clinical rapid detection.

To solve above problems, the technical proposal of the invention is as follows: a NIR-II fluorescent rare earth nanoprobe test strip, includes a plastic backing (i.e., bottom plate), a sample pad, a conjugation pad, a nitrocellulose (NC) membrane, and an absorbent pad; the sample pad, the conjugation pad, the NC membrane, and the absorbent pad are superimposed on the plastic backing successively along a horizontal direction; detection antibodies labeled with RENPs are immobilized on the conjugation pad; capture antibodies as a test line (T line) and quality control antibodies as a control line (C line) are sprayed on the NC membrane.

Based on above technical proposal, the invention can also make the following improvements:

In an embodiment, the nanoprobe is a kind of RENPs with fluorescence emission peaks in the NIR-II region.

In an embodiment, a fluorescent emission wavelength of RENPs ranges from 900 nm to 1700 nm. An excitation wavelength of RENPs ranges from 700 nm to 900 nm.

In an embodiment, the nanoprobe (i.e., NIR-II fluorescent rare earth nanoprobe test strip) is doped by any one or more rare earth ions in Nd³⁺, Yb³⁺, Er³⁺, Ho³⁺ or Ce³⁺.

In an embodiment, the nanoprobe includes an inner core and an outer shell. The inner core is an inorganic matrix doped ion, including NaLnF₄, LiLnF₄ or LnF₃, where Ln=Y, Yb, Gd or Lu.

The outer shell is one or more inorganic substrates coated outside the kernel, including NaLnF₄, LiLnF₄, or CaF₂, where Ln═Y, Yb, Gd, or Lu.

In an embodiment, the nanoprobe is a kind of the surface functionalized RENPs, which is surface-modified with one or several high molecular polymers, including sodium citrate, polyacrylic acid (PAA), phospholipid polyethylene glycol carboxyl group (i.e., distearoyl phosphoethanolamine-polyethylene glycol-carboxyl, DSPE-PEG-COOH), poly(lactide-co-glycolide)-b-poly(ethylene glycol)-carboxylic acid (i.e., poly-dl-lactic-co-glycolic-polyethylene glycol-carboxyl, PLGA-PEG-COOH), poly(maleic anhydride-alt-1-octadecene) (PMA-ODE), poly(ε-caprolactone)-poly(ethylene glycol)-acid (PCL-PEG-COOH), and polyethylene glycol-polylactic acid-carboxyl (PEG-PLA-COOH). A particle size of the RENPs is in a range of 20 nm to 200 nm.

The invention also discloses a preparation method of a NIR-II fluorescent rare earth nanoprobe test strip as described above, which includes the following steps:

(1) Preparation of Sample Pad

The glass fiber or the polyester fiber is chosen and soaked in the sample pad treatment solution, and then shaking at room temperature (RT) for 2-3 hours (h), and drying in the oven;

(2) Preparation of Conjugation Pad

The RENPs labeling solution is diluted with a diluent (i.e., microsphere diluent) to obtain a diluted RENPs labeling solution, and then the diluted RENPs labeling solution is evenly spread on the glass fiber or the polyester fiber, followed by drying in the oven;

(3) Coating Antibody of NC Membrane

Capture antibody solution and quality control antibody solution are firstly prepared with a phosphate buffer saline (PBS) buffer, respectively. Capture antibodies (i.e., the capture antibody solution) set as the test line (T line) and quality control antibody (i.e., quality control antibody solution) set as the control line (C line) are sprayed on the NC membrane by a three dimensional point spray platform, then drying in the oven (such as in the oven at 45° C.);

(4) Preparation of Absorbent Pad

The absorbent pad is cut to the size of 30*2.5 cm (i.e., target size);

(5) Assembling of Detection Card

The prepared sample pad, conjugation pad, NC membrane, absorbent pad are superimposed on the plastic backing successively along the horizontal direction. Test strips are prepared with an automatic cutting machine, then putting them into the card slots to make the detection cards, finally the detection cards are stored in dry environment.

In an embodiment, the preparation of RENPs labeling solution in step 2 includes the following process:

\textcircled{1} Activating Treatment of RENPs

Surface carboxylated RENPs are incubated with 1-(3-Dimethylaminopropyl)-3-ethylcarbodiimide hydro (EDC) and n-hydroxy succinimide (NHS) in 2-morpholinoethanesulphonic acid (MES) buffer (50 mM, pH 6.0) at the room temperature RT for 30 min. After centrifugation, the precipitate is fully dispersed by ultrasound. Specifically, ultrasonic resuspension is performed on the carboxylated RENPs, and then a supernatant is discarded after high-speed centrifugation to obtain a precipitate; the MES buffer is added into the precipitate for ultrasonically dispersing, followed by adding EDC and Sulfo-NHS, oscillating and performing a reaction to obtain a first product; a supernatant of the first product is discarded and a precipitate of the first product is obtained after high-speed centrifugation, followed by adding a phosphate buffer (PB) for ultrasonic dispersion to obtain an activated RENPs solution;

\textcircled{2} Conjugation Antibody of RENPs

The activated RENPs are incubated with the detection antibody at RT for 2 h, followed by adding into 10% bovine serum albumin (BSA) solution to block conjugation reaction. After centrifugation, the precipitate is treated with washing buffer, followed by adding with the protective solution, finally storing at 4° C. Specifically, the detection antibodies are added into the activated RENPs solution for oscillating, then the BSA solution is added for sealing, and finally the ethanolamine solution is added to terminate the reaction to obtain a second product; a supernatant of the second product is discarded and a precipitate of the second product is obtained after high-speed centrifugation, followed by adding microsphere washing liquid into the precipitate of the second product and ultrasonically suspending for 2-3 times; then adding a microsphere protective solution and storing at 4° C. for later use.

In an embodiment, the molar ratio of the carboxylated RENPs, EDC and NHS (i.e., Sulfo-NHS) in step \textcircled{1} is 1:5:10.

In an embodiment, the microsphere washing solution is 10 millimoles per liter (mM) phosphate buffer saline (PBS) buffer containing 0.5% BSA and 0.1% Tween-20, and the composition of microsphere protective solution is 10 mM PBS buffer containing 0.5% BSA.

In an embodiment, the microsphere diluent is 10 mM citric acid solution containing 1% sucrose.

In an embodiment, the treatment solution of sample pad is 20 mM tris(hydroxymethyl)aminomethane (Tris) buffer containing 0.5% surfactant S9 (i.e., Tetronic™ 1307), 0.05% BSA, 0.05% Tween-20, 0.3% polyvinylpyrrolidone K30 (PVP-K30) and 0.05% PROCLIN-300.

In an embodiment, the addition of antibody in step \textcircled{2} is 0.05-0.2 mg.

In an embodiment, the concentrations of T line and C line in step (3) are 0.5-2.0 mg·mL⁻¹.

In an embodiment, a spraying amount of the T line and the C line is 0.5-2 μL·cm⁻¹.

Compared with the existing rapid test strip, the invention has the following advantages:

The invention chooses RENPs with NIR-II fluorescence as an efficient probe, and its excellent optical properties make the prepared test strip has excellent detection sensitivity, good accuracy, high stability and favorable repeatability.

The preparation method of NIR-II test strip has advantages of simplicity and low price, which is suitable for large-scale production of test strip.

### BRIEF DESCRIPTION OF DRAWINGS

FIG. 1 illustrates a schematic structural diagram of a prepared NIR-II fluorescent test strip.

FIG. 2 illustrates a photo taken by InGaAs camera based on the prepared NIR-II fluorescent test strip for detecting CEA.

FIG. 3 illustrates a standard curve of the prepared NIR-II fluorescent test strip for detecting CEA.

FIG. 4 illustrates a detection result comparison of NIR-II fluorescence method and the Abbott chemiluminescence immunoassay method.

Description of reference numerals: 1. plastic backing; 2. sample pad; 3. conjugation pad; 4. NC membrane; 5. absorbent pad; 6. test line; 7. control line.

### DETAILED DESCRIPTION OF EMBODIMENTS

The principles and features of the invention are described in combination with the attached drawings. The embodiments given are only used to explain the invention and are not used to limit the scope of the invention.

As shown in FIG. 1, a NIR-II fluorescent rare earth nanoprobe test strip, include: a plastic backing 1, a sample pad 2, a conjugation pad 3, a NC membrane 4 and an absorbent pad 5. The sample pad 2, the conjugation pad 3, the NC membrane 4, and the absorbent pad 5 are superimposed on the plastic backing 1 successively along a horizontal direction. CEA detection antibodies labeled RENPs are immobilized on the conjugation pad 3. CEA capture antibodies set as a test (T) line 6 and IgG as a control (C) line 7 are sprayed on the NC membrane 4.

The probe possesses fluorescent emission double peaks, and the central wavelength of the emission peak is 1064±15 nm and 1345±15 nm, respectively.

The structural component is NaYF₄:X % Nd@NaYF₄, X=1-100.

The particle size of the RENPs is 20-200 nm.

### Embodiment 1

Preparation of RENPs Labeling Solution

The labeling process of RENPs and CEA detection antibody consist of the following two steps:

(1) Activating Treatment of RENPs

0.1 mL RENPs-COOH and 1 mL MES buffer (50 mM, pH 6.0) are mixed for 2 min by ultrasound, then centrifuged at 14000 rpm for 15 min, followed by discarding the supernatant. The precipitate is resuspended in 1 mL MES buffer by ultrasound, then the freshly prepared 0.1 mL EDC solution (10 mg mL−1) and 0.2 mL NHS solution (10 mg mL−1) are added. The mixed solution is placed at roller mixer for 30 min, then centrifuged at 14000 rpm for 15 min, then discarding the supernatant. The precipitate is resuspended in 0.5 mL PBS buffer (10 mM, pH 7.4) by ultrasound.

(2) Conjugation CEA Detection Antibody of RENPs

The activated RENPs are incubated with 0.15 mg CEA detection antibody at RT for 2 h, followed by adding into 0.1 mL 10% BSA to block conjugation reaction, then centrifuging at 14000 rpm for 15 min, and discarding the supernatant. The precipitate is treated with washing buffer (10 mM PBS buffer containing 0.5% BSA and 0.1% Tween-20), followed by adding with the protective solution (10 mM PBS buffer containing 0.5% BSA), finally storing at 4° C.

### Embodiment 2

The Preparation Method of a NIR-II Fluorescent Rare Earth Nanoprobe Test Strip for Detection of CEA

A NIR-II fluorescent rare earth nanoprobe test strip, include a plastic backing 1, a sample pad 2, a conjugation pad 3, a NC membrane 4, an absorbent pad 5. The adjacent parts overlap each other by 2 mm on the plastic backing 1.

The Preparation Method is as Follows:

(1) Preparation of Sample Pad 2

The glass fiber is chosen and soaked in the sample pad treatment solution (20 mM Tris buffer containing 0.5% S9, 0.05% BSA, 0.05% Tween-20, 0.3% PVP-K30 and 0.05% Proclin-300), and then shaking at room temperature for 2 h, drying in the oven at 45° C. for 24 h;

(2) Preparation of Conjugation Pad 3

The diluent (10 mM citric acid solution containing 1% sucrose) is added into RENPs labeling solution to dilute to 20% of the original concentration to obtain a diluted RENPs labeling solution, and then the diluted RENPs labeling solution is evenly spread on the glass fiber, and then drying in the oven at 45° C. for 24 h.

(3) Coating Antibody of NC Membrane 4

2 mg·mL⁻¹ CEA capture antibody solution is prepared by 10 mM PBS buffer (pH is 7.4). It is sprayed on the NC membrane as the T line 6 by a three dimensional point spray platform with a spraying amount of 1.2 μL/cm. The lining speed is 50 mm/s, and it is dried in the oven at 45° C. for 12 hours.

2 mg·mL⁻¹ IgG solution is prepared by 10 mM PBS buffer (pH is 7.4). It is sprayed on the NC membrane as the C line 7 by a three dimensional point spray platform with a spraying amount of 1.2 μL/cm, then drying in the oven at 45° C. for 12 h. The distance between line 7 and line 6 is 5 mm. The lining speed is 50 mm/s.

(4) Preparation of Absorbent Pad 5

The absorbent pad is cut to the size of 30*2.5 cm.

(5) Assembling of Detection Card

The prepared sample pad 2, conjugation pad 3, NC membrane 4, absorbent pad 5 are superimposed on the plastic backing 1 successively along the horizontal direction. 4 mm wide test strips are prepared with an automatic cutting machine, then put them into the card slot, and making detection cards, finally storing in dry environment.

### Embodiment 3

Detection Card from Embodiment 2 for Detection of CEA

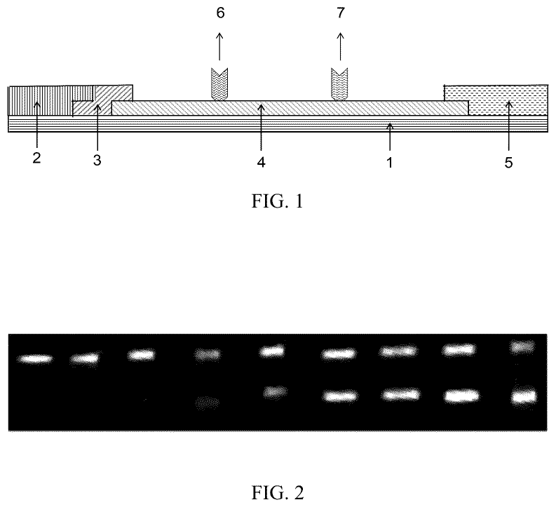

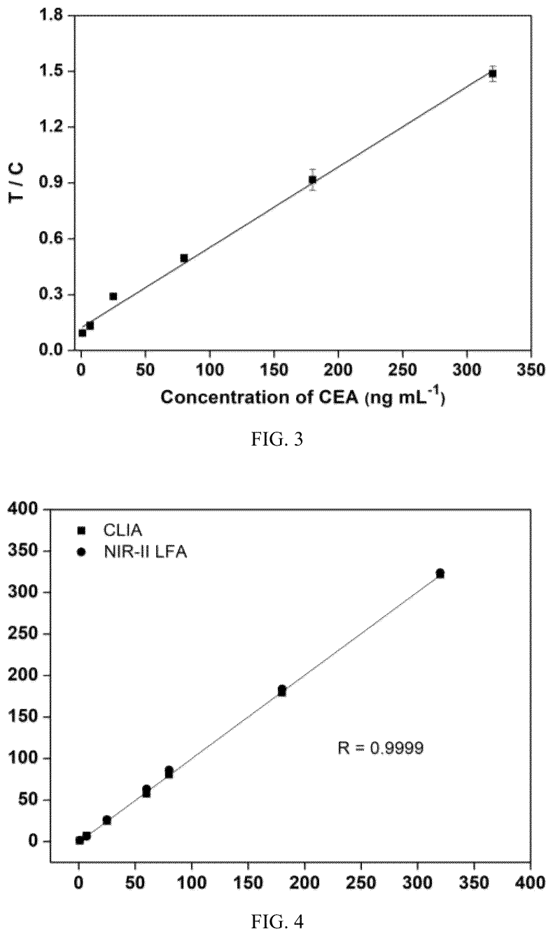

CEA serum quality control and detection cards from embodiment 2 are restored to RT. 40 μL CEA serum quality control with concentrations of 0, 1.0, 7.0, 25.0, 80.0, 180.0 and 320.0 ng·mL⁻¹ are added into the sampling well of detection card, respectively. Then, 40 μL sample diluent is added. After 15 min, detection cards are placed in the NIR-II imaging system to take picture (see FIG. 2). The fluorescence intensity of T line and C line are recorded, then T/C ratio is calculated. The standard curve based on CEA concentrations and T/C ratio is established (see FIG. 3). Each concentration point is tested 3 times in parallel.

The prepared detection cards are used to test concentrations of 14 clinical samples, which have been confirmed by Abbott chemiluminescence immunoassay. The calculated T/C ratio is plugged in the standard curve equation to obtain sample concentration results. Then, the correlations between two methods are compared.

The experimental results show that two methods have good consistency (see FIG. 4), indicating that the NIR-II test strip in this invention has excellent accuracy and reliability for the CEA detection of clinical samples.

The above is only a better embodiment of the invention and is not intended to limit the invention, and any modification, equivalent replacement, improvement, etc. made within the spirit and principles of the invention shall be included in the scope of protection of the invention.

What is claimed is:

1. A near infrared-II region (NIR-II) fluorescent rare earth nanoprobe test strip, comprising five parts: a sample pad, a conjugation pad, a nitrocellulose (NC) membrane, an absorbent pad and a plastic backing;
wherein the sample pad, the conjugation pad, the NC membrane, and the absorbent pad are superimposed on the plastic backing successively along a horizontal direction; detection antibodies labeled with rare earth nanoparticles (RENPs) are immobilized on the conjugation pad; and capture antibodies as a test line and quality control antibodies as a control line are sprayed on the NC membrane.

2. The NIR-II fluorescent rare earth nanoprobe test strip as claimed in claim 1, wherein the NIR-II fluorescent rare earth nanoprobe test strip is a kind of RENPs with fluorescent emission peak in a NIR-II region.

3. The NIR-II fluorescent rare earth nanoprobe test strip as claimed in claim 2, wherein the RENPs are doped by one or more of rare earth ions.

4. The NIR-II fluorescent rare earth nanoprobe test strip as claimed in claim 3, wherein the RENPs comprise an inner core and an outer shell.

5. The NIR-II fluorescent rare earth nanoprobe test strip as claimed in claim 4, wherein a surface of the RENPs is modified with one or more of molecular polymers.

6. The NIR-II fluorescent rare earth nanoprobe test strip as claimed in claim 2, wherein a structure of the NIR-II fluorescent rare earth nanoprobe test strip probe comprises: NaYF₄: X % Nd@NaYF₄, and X=1-100.

7. The NIR-II fluorescent rare earth nanoprobe test strip as claimed in claim 6, wherein the NIR-II fluorescent rare earth nanoprobe test strip probe is a kind of carboxylated RENPs with a surface modified by one or more of sodium citrate, polyacrylic acid (PAA), distearoyl phosphoethanolamine-polyethylene glycol-carboxyl (DSEP-PEG-COOH), poly-dl-lactic-co-glycolic-polyethylene glycol-carboxyl (PLGA-PEG-COOH), and polyethylene glycol-polylactic acid-carboxyl (PEG-PLA-COOH); and a particle size of the RENPs is in a range of 20 nm to 200 nm.

8. A preparation method of the NIR-II fluorescent rare earth nanoprobe test strip as claimed in claim 1, comprising the following steps:
(1) preparation of the sample pad:
choosing one of a glass fiber and a polyester fiber, soaking the one of the glass fiber and the polyester fiber in a sample pad treatment solution, then shaking at room temperature (RT) for 2-3 hours (h), and finally drying in an oven;
(2) preparation of the conjugation pad:
diluting a RENPs labeling solution with a microsphere diluent to obtain a diluted RENPs labeling solution, evenly spreading the diluted RENPs labeling solution on the one of the glass fiber and the polyester fiber, and then drying in the oven;
(3) coating antibody of the NC membrane:
preparing a capture antibody solution and a quality control antibody solution with a buffer, respectively; simultaneously spraying the capture antibody solution and the quality control antibody solution on the NC membrane to form the capture antibodies set as the test line (T line) and the quality control antibodies as the control line (C line) on the NC membrane with a three dimensional point spray platform, and then drying in the oven;
(4) preparation of the absorbent pad:
cutting the absorbent pad to a target size;
(5) assembling of a detection card:
superimposing the sample pad, the conjugation pad, the NC membrane, the absorbent pad on the plastic backing successively along the horizontal direction; preparing test strips with an automatic cutting machine, then putting the test strips into card slots to make detection cards, finally storing the detection cards in a dry environment.

9. The preparation method as claimed in claim 8, wherein a preparation of the RENPs labeling solution in the step (2) comprises the following steps:
\textcircled{1} activating treatment of RENPs:
performing ultrasonic resuspension on carboxylated RENPs, and then discarding a supernatant after high-speed centrifugation to obtain a precipitate;
adding a 2-morpholinoethanesulphonic acid (MES) buffer into the precipitate, ultrasonically dispersing the precipitate added with the MES buffer, followed by adding an activator of 1-(3-Dimethylaminopropyl)-3-ethylcarbodiimide hydro (EDC) and a coupling agent of N-hydroxysulfosuccinimide (Sulfo-NHS), oscillating and performing a reaction to obtain a first product; discarding a supernatant of the first product and obtaining a precipitate of the first product after high-speed centrifugation, followed by adding a phosphate buffer for ultrasonic dispersion to obtain an activated RENPs solution;
\textcircled{2} conjugation antibody of RENPs:
adding the detection antibodies into the activated RENPs solution, oscillating for a reaction, then adding a bovine serum albumin (BSA) solution for sealing, and finally adding an ethanolamine solution to terminate the reaction to obtain a second product; discarding a supernatant of the second product and obtaining a precipitate of the second product after high-speed centrifugation, followed by adding microsphere washing liquid into the precipitate of the second product and ultrasonically suspending for 2-3 times; then adding a microsphere protective solution and storing at 4° C. for later use.

10. The preparation method as claimed in claim 9, wherein in the step \textcircled{1}, a molar ratio of the carboxylated RENPs: the EDC: the Sulfo-NHS is 1:5:10.

11. The preparation method as claimed in claim 9, wherein in the step \textcircled{2}, the microsphere washing solution is a 10 millimoles per liter (mM) phosphate buffer saline (PBS) buffer containing 0.5% BSA and 0.1% Tween-20, and the microsphere protective solution is a 10 mM PBS buffer containing 0.5% BSA.

12. The preparation method as claimed in claim 8, wherein the microsphere diluent comprises a 10 mM citric acid solution containing 1% sucrose.

13. The preparation method as claimed in claim 8, wherein the sample pad treatment solution comprises a 20 mM tris(hydroxymethyl)aminomethane (Tris) buffer containing 0.5% surfactant, 0.05% BSA, 0.05% Tween-20, 0.3% polyvinylpyrrolidone K30 (PVP-K30) and 0.05% PROCLIN-300.
